(19) United States
(12) Patent Application Publication
(10) Pub. No.: US 2026/0364082 A1
(43) Pub. Date: Nov. 19, 2026

(54) METHODS OF TREATING DERMATOLOGICAL DISORDERS

(71) Applicant: Alumis Inc., South San Francisco, CA (US)

(72) Inventors: Mera Tilley, South San Francisco, CA (US); Joshua Hoffman, South San Francisco, CA (US); Claire Langrish, South San Francisco, CA (US)

(21) Appl. No.: 19/157,136

(22) PCT Filed: Feb. 16, 2024
(86) PCT No.: PCT/US2024/016103

§ 371 Date: Aug. 15, 2025

Related U.S. Application Data
(60) Provisional application No. 63/485,409, filed on Feb. 16, 2023.

(51) Int. Cl. A61K 31/5377; A61K 31/4985; A61K 31/501; A61K 31/506; A61K 31/519; A61K 31/55; C12Q 1/6883

(52) U.S. Cl. CPC A61K 31/5377; A61K 31/4985; A61K 31/501; A61K 31/506; A61K 31/519; A61K 31/55; C12Q 1/6883; C12Q 2600/158

(57) ABSTRACT

The disclosure is directed, in part, to methods of treating a patient suffering from a TYK2-mediated disorder, e.g., an auto-immune skin disease or a dermatological disorder disclosed herein, comprising administering to the patient and effective amount of a TYK2 inhibitor.

### CROSS REFERENCE TO RELATED APPLICATIONS

This application claims the benefit of, and priority to, U.S. Provisional Patent Application No. 63/485,409, filed on Feb. 16, 2023; the content of which is hereby incorporated by reference herein in its entirety.

### BACKGROUND

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g., IFN-α, IFN-β, IFN-κ, IFN-δ, IFN-ε, IFN-τ, IFN-ω, and IFN-ζ (also known as limitin), and interleukins (e.g., IL-4, IL-6, IL-10, IL-11, IL-12, IL-13, L-22, IL-23, IL-27, IL-31, oncostatin M, ciliary neurotrophic factor, cardiotrophin 1, cardiotrophin-like cytokine, and LIF). The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. A genome-wide association study of 2,622 individuals with psoriasis identified associations between disease susceptibility and TYK2. Knockout or tyrphostin inhibition of TYK2 significantly reduces both IL-23 and IL-22-induced dermatitis.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Genome-wide association studies of individuals with SLE versus an unaffected cohort showed highly significant correlation between the TYK2 locus and SLE.

Accordingly, there is a need to provide methods for inhibiting the activity of TYK2 to treat one or more of the conditions described herein, without the side-effects associated with the inhibition of JAK2.

### SUMMARY

The disclosure is directed, in part, to methods of treating TYK2-mediated disorders, e.g., an auto-immune skin disease or a dermatological disorder. For example, provided herein is a method of treating a patient suffering from an auto-immune skin disease and having elevated Type I IFN induced sialic acid-binding immunoglobulin-like lectin1 (SIGLEC1), and in need of treatment, comprising administering to the patient an effective amount of a TYK2 inhibitor.

Also disclosed herein is a method of treating a dermatological disorder in a patient having elevated Type I IFN-induced sialic acid-binding immunoglobulin-like lectin 1 (SIGLEC1) expression, and in need of treatment, comprising administering to the patient an effective amount of a TYK2 inhibitor, and thereby upon administration reducing the expression of SIGLEC1 in the patient.

Further described herein, for example, is a method of reducing expression of SIGLEC1 protein or gene in a cell, comprising contacting the cell with an agent that reduces TYK2 gene expression or reduces TYK2 in the cell, thereby reducing the expression of SIGLEC1.

In addition, provided herein is a method of treating a subject suffering from an auto-immune skin disease, and in need of treatment, comprising: providing a biological sample from the subject; assaying gene expression of one or more IFN-1 genes in the biological sample; determining the expression value of the IFN-1 gene(s); and if the expression value is above a threshold value, administering a TYK2 inhibitor to the patient.

### BRIEF DESCRIPTION OF THE DRAWINGS

FIG. 1 depicts a median IFN signature violin plot of median IFN gene expression in disease (HS) and control states.

FIG. 2 depicts a median IFN signature density plot of median IFN gene expression in disease (HS) and control states.

FIG. 3 depicts a density plot of SIGLEC1 gene expression in disease (HS) and control states.

FIG. 4 shows dose-dependent median IFN gene expression levels over time for HERC5, IFI27, IFIT1, RSAD2 in healthy volunteers after 14 days Compound A versus placebo.

FIG. 5 shows dose-dependent median IFN gene expression levels (log scale) over time for HERC5, IFI27, IFIT1, RSAD2 in healthy volunteers after 14 days Compound A versus placebo.

FIG. 6 shows dose-dependent median IFN gene expression levels over time for SIGLEC1 in healthy volunteers after 14 days Compound A versus placebo.

FIG. 7 shows dose-dependent median IFN gene expression levels (log scale) over time for SIGLEC1 in healthy volunteers after 14 days Compound A versus placebo.

FIG. 8 shows percent change in PASI score as a function of baseline SIGLEC1 gene expression (log 10 scale) in patients with psoriasis after 12 weeks of treatment with placebo.

FIG. 9 shows percent change in PASI score as a function of baseline SIGLEC1 gene expression (log 10 scale) in patients with psoriasis after 12 weeks of treatment with compound A, 10 mg, once daily.

FIG. 10 shows percent change in PASI score as a function of baseline SIGLEC1 gene expression (log 10 scale) in patients with psoriasis after 12 weeks of treatment with compound A, 20 mg, once daily.

FIG. 11 shows percent change in PASI score as a function of baseline SIGLEC1 gene expression (log 10 scale) in patients with psoriasis after 12 weeks of treatment with compound A, 40 mg, once daily.

FIG. 12 shows percent change in PASI score as a function of baseline SIGLEC1 gene expression (log 10 scale) in patients with psoriasis after 12 weeks of treatment with compound A, 20 mg, twice daily.

FIG. 13 shows percent change in PASI score as a function of baseline SIGLEC1 gene expression (log 10 scale) in patients with psoriasis after 12 weeks of treatment with compound A, 40 mg, twice daily.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### DEFINITIONS

As used herein and in the appended claims, the singular forms “a,” “an,” and “the” include plural referents unless the context clearly dictates otherwise. Thus, for example, reference to “an agent” includes a plurality of such agents, and equivalents thereof known to those skilled in the art, and so forth. When ranges are used herein, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound or composition disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

### Methods

Disclosed herein, for example, is a method of treating a patient suffering from an auto-immune skin disease and having elevated Type I IFN induced sialic acid-binding immunoglobulin-like lectin1 (SIGLEC1), and in need of treatment, comprising administering to the patient an effective amount of a TYK2 inhibitor.

In some embodiments, a lesional skin sample from the patient before administration of the TYK2 inhibitor has elevated Type I IFN-induced sialic acid-binding immunoglobulin-like lectin1 (SIGLEC1) expression. In certain embodiments, the elevated Type I IFN induced sialic acid-binding immunoglobulin-like lectin1 (SIGLEC1) expression is elevated as compared to the SIGLEC1 expression in the whole blood of the patient, or as compared to a patient not suffering from the auto-immune skin disease. In further embodiments, a biological sample from the patient before administration of the TYK2 inhibitor has increased IFN-1 gene expression. For example, in certain embodiments the increased IFN-1 gene expression is one or more of increased gene expression of: OAS3, OAS2, CD8A, EPSTI1, BST2, RNF213, EIF2AK2, IFIT2, IFIT3, SP100, SP110, LY6E, MX1, IFI6, RTP4, XAF1, PATL2, STAT1, and SIGLEC1.

Also disclosed herein is a method of treating a dermatological disorder in a patient having elevated Type I IFN-induced sialic acid-binding immunoglobulin-like lectin1 (SIGLEC1) expression, and in need of treatment, comprising administering to the patient an effective amount of a TYK2 inhibitor, and thereby upon administration reducing the expression of SIGLEC1 in the patient.

Further disclosed herein is a method of reducing expression of SIGLEC1 protein or gene in a cell, comprising contacting the cell with an agent that reduces TYK2 gene expression or reduces TYK2 in the cell, thereby reducing the expression of SIGLEC1. In some embodiments, cell is, for example, a skin cell.

In addition, disclosed herein is a method of treating a subject suffering from an auto-immune skin disease, and in need of treatment, comprising: providing a biological sample from the subject; assaying gene expression of one or more IFN-1 genes in the biological sample; determining the expression value of the IFN-1 gene(s); and if the expression value is above a threshold value, administering a TYK2 inhibitor to the patient. For example, in some embodiments, the IFN-1 gene is one or more of: OAS3, OAS2, CD8A, EPSTI1, BST2, RNF213, EIF2AK2, IFIT2, IFI6, and SIGLEC1. In certain embodiments, the biological sample is, e.g., the subject's blood or tissue.

In addition, disclosed herein is a method of treating a subject suffering from an auto-immune skin disease, and in need of treatment, comprising: providing a biological sample from the subject; assaying protein expression of one or more IFN-1 proteins in the biological sample; determining the expression value of the IFN-1 protein(s); and if the expression value is above a threshold value, administering a TYK2 inhibitor to the patient.

In some embodiments, the TYK2 inhibitor is selected from, e.g., the group consisting of, for example, GLPG3121, GLPG3667, VTX-958, ICP-332, BGB-23339,

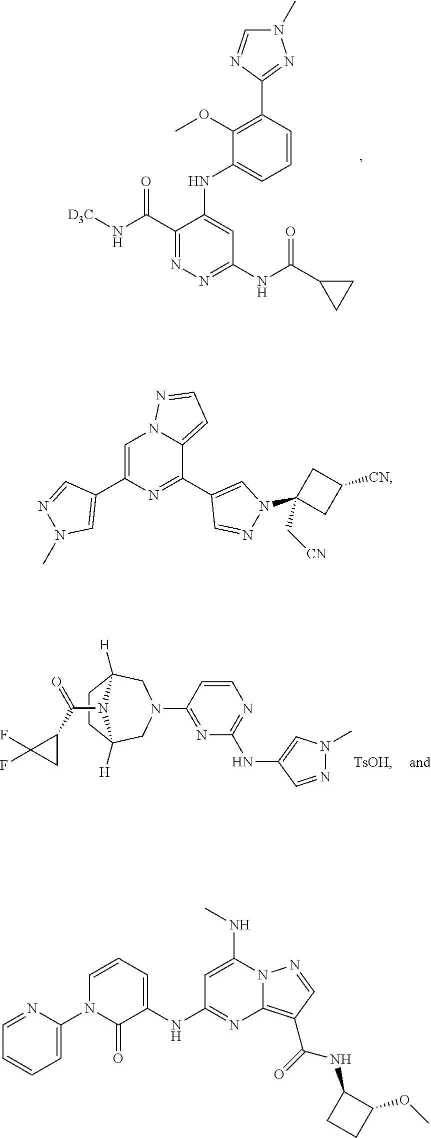 [SMILES: [2H]C([2H])([2H])NC(=O)c1nnc(NC(=O)C2CC2)cc1Nc1cccc(-c2ncn(C)n2)c1OC ; Cn1cc(-c2cn3nccc3c(-c3cnn([C@]4(CC#N)C[C@@H](C#N)C4)c3)n2)cn1 ; Cn1cc(Nc2nccc(N3C[C@H]4CC[C@@H](C3)N4C(=O)[C@@H]3CC3(F)F)n2)cn1 ; CNc1cc(Nc2cccn(-c3ccccn3)c2=O)nc2c(C(=O)N[C@@H]3CC[C@H]3OC)cnn12]

In other embodiments, the TYK2 inhibitor is represented by:

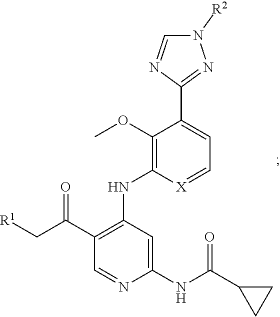

or a pharmaceutically acceptable salt thereof; wherein X is CH or N; and R¹ and R² are independently for each occurrence selected from CH₃ and CD₃. In certain embodiments, the methods described herein comprise orally administering or topically administering the compound to the patient.

For example, in some embodiments a disclosed TYK2 inhibitor may be selected from the group consisting of, e.g.,

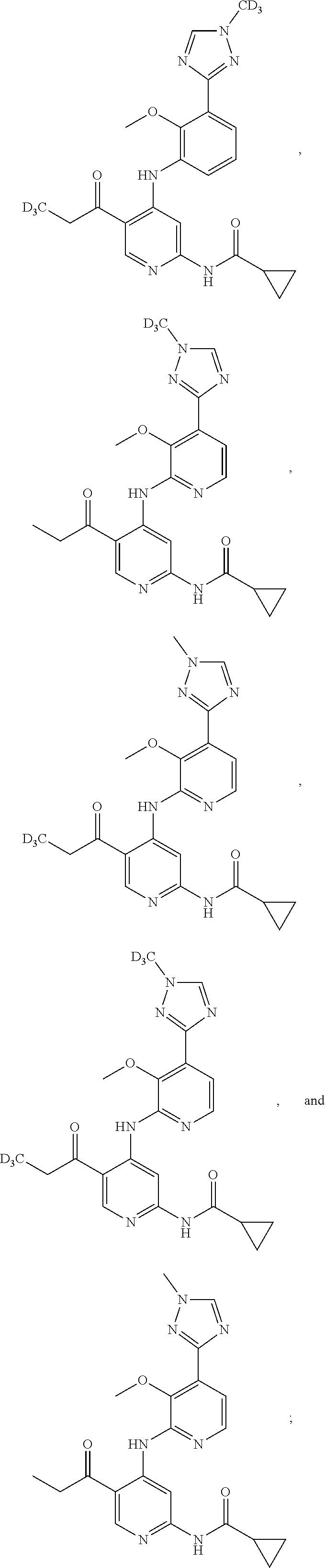 [SMILES: [2H]C([2H])([2H])CC(=O)c1cnc(NC(=O)C2CC2)cc1Nc1cccc(-c2ncn(C([2H])([2H])[2H])n2)c1OC ; [2H]C([2H])([2H])n1cnc(-c2ccnc(Nc3cc(NC(=O)C4CC4)ncc3C(=O)CC)c2OC)n1 ; [2H]C([2H])([2H])CC(=O)c1cnc(NC(=O)C2CC2)cc1Nc1nccc(-c2ncn(C)n2)c1OC ; [2H]C([2H])([2H])CC(=O)c1cnc(NC(=O)C2CC2)cc1Nc1nccc(-c2ncn(C([2H])([2H])[2H])n2)c1OC ; CCC(=O)c1cnc(NC(=O)C2CC2)cc1Nc1nccc(-c2ncn(C)n2)c1OC]

or a pharmaceutically acceptable salt thereof.

In further embodiments, the auto-immune skin disease or dermatological disorder is, for example, one or more of: systemic sclerosis, Sjogren's syndrome, cutaneous lupus erythematosus, systemic lupus erythematosus with skin manifestations, psoriasis, dermatomyositis, hidradenitis suppurativa, vitiligo, and Behcet's Disease. In other embodiments, the auto-immune skin disease or dermatological disorder is one or more of, for example: systemic sclerosis, Sjogren's syndrome, cutaneous lupus erythematosus, systemic lupus erythematosus with skin manifestations, psoriasis, dermatomyositis, hidradenitis suppurativa, vitiligo, and Behcet's Disease.

For example, provided herein are methods for treating an inflammatory or allergic condition of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

In some embodiments, the methods described herein comprise orally administering or topically administering the compound to the patient, for example, orally administering 10 mg to 80 mg of the compound once or twice daily; for example, orally administering 40 mg or 60 mg of the compound once or twice daily. In other embodiments, the methods described herein further comprise administering an antibiotic to the patient.

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration (e.g., oral administration) and standard pharmaceutical practice as described, for example, in Remington: The Science and Practice of Pharmacy (Gennaro, 21^{st} Ed. Mack Pub. Co., Easton, PA (2005)).

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

For example, in some embodiments a disclosed compound or composition may be administered once daily to the patient. In other embodiments, a disclosed compound or composition may be administered twice daily to the patient.

In some embodiments, a disclosed compound or pharmaceutical composition is formulated for oral administration. In some embodiments, the pharmaceutical composition is formulated as a tablet, a pill, a capsule, a liquid, a suspension, a dispersion, a solution, or an emulsion. In some embodiments, the pharmaceutical composition is formulated as a tablet, for example, a tablet formulated for oral administration.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involve the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

Further, by way of example only, the dose of the compound for methods of treating a disease as described herein may be a fixed dose, e.g., a fixed dose of 10 mg, 15 mg, 20 mg, 25 mg, 30 mg, 35 mg, 40 mg, 45 mg, 50 mg, 55 mg, 60 mg, 65 mg, 70 mg, 75 mg, or 80 mg.

In certain instances, a compound described herein is administered in combination with a second therapeutic agent. In some embodiments, the benefit experienced by a patient is increased by administering a compound described herein with a second therapeutic agent that also has therapeutic benefit. In one specific embodiment, a compound described herein is co-administered with a second therapeutic agent wherein the compound described herein and the second therapeutic agent modulate different aspects of the disease, disorder or condition being treated, thereby providing a greater overall benefit than administration of either therapeutic agent alone. The overall benefit experienced by the patient is simply additive of the two therapeutic agents or the patient experiences a synergistic benefit.

It is understood that the dosage regimen to treat, prevent, or ameliorate the condition(s) for which relief is sought, is modified in accordance with a variety of factors (e.g., the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

The compounds describe herein are administered before, during, or after the occurrence of a disease or condition, and the timing of administering the composition containing a compound varies. Thus, in one embodiment, the compounds and compositions described herein are used as a prophylactic and are administered continuously to subjects with a propensity to develop conditions or diseases in order to prevent the occurrence of the disease or condition. In another embodiment, the compounds are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only, and are not intended to limit the scope of the disclosure.

### Example 1: Differential Gene Expression (DGE) Analysis in a Case Control HS Dataset

### Analytical Methods

A differential gene expression (DGE) analysis in a case control HS dataset was conducted. The dataset used in the analysis was imported from NCBI's BioProjects under accession number PRJNA647407. The dataset was contributed by “Prens E P, Gudjonsson J E, Tsoi L C” for hidradenitis suppurativa. The dataset was a case control set consisting of transcriptome profiling in lesional tissue as well as whole blood. The analytical dataset was raw counts at the gene level. Custom quantification was performed using STAR->Salmon->RSEM. Differential expression analysis was carried using a DESeq2 program for normalization, visualization, and analysis of RNA-seq data, comparing cases to controls. A geometric mean was calculated for each gene across all samples. The counts for a gene in each sample was then divided by this mean, with the median of these ratios in a sample being the size factor for that sample. Genes were removed that contained less than 20 counts summed across all individuals.

### Results

Differential gene expression analysis was also conducted on interferon type-1 (IFN-1) cytokine genes. Table 1 shows the top differentially expressed IFN-1 genes in lesional tissue and whole blood samples obtained from patients with hidradenitis suppurativa. As shown in Table 1, Type I IFN induced sialic acid-binding immunoglobulin-like lectin 1 (SIGLEC1) expression is elevated as compared to the SIGLEC1 expression in patients having hidradenitis suppurativa as compared to base levels.

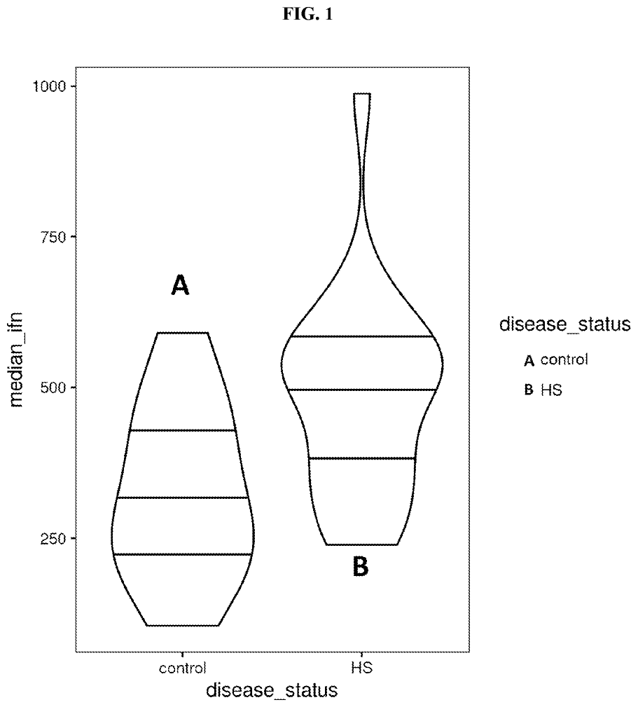

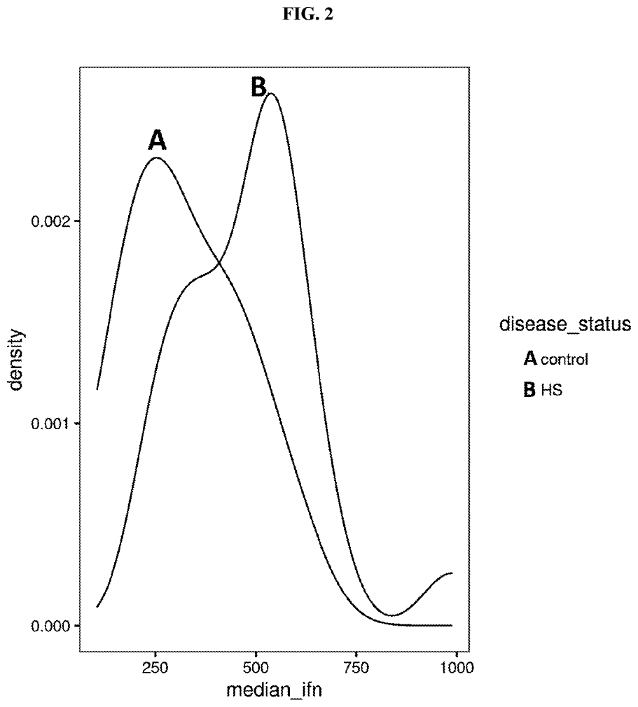

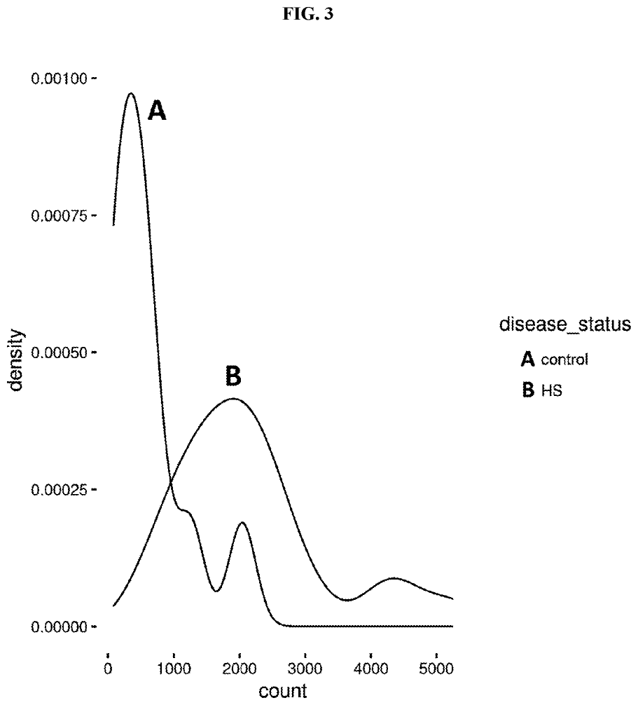

FIG. 1 depicts a median IFN signature violin plot of median IFN gene expression in disease and control states. As shown if FIG. 1, subjects having hidradenitis suppurativa displayed increased IFN gene expression (shown as median) compared to non-disease status. FIG. 2 depicts a median IFN signature density plot of median IFN gene expression in disease and control states. Similar to FIG. 1, FIG. 2 also indicates that subjects having hidradenitis suppurativa displayed an increase in IFN expression (shown as median) compared to non-disease status. FIG. 3 depicts a density plot of SIGLEC1 gene expression in disease and control states. As shown in FIG. 3, an increase in median SIGLEC1 expression was observed in subjects hidradenitis suppurativa relative to non-disease state.

TABLE 1

| Gene symbol | baseMean | log2FoldChange | lfcSE | stat | pvalue | Padj |
|---|---|---|---|---|---|---|
| CD8A | 559.5 | 2.027 | 0.315 | 6.434 | 1.24e−10 | 3.103e−09 |
| OAS2 | 1616 | 1.732 | 0.2692 | 6.434 | 1.244e−10 | 3.108e−09 |
| BST2 | 1332 | 1.654 | 0.2576 | 6.419 | 1.37e−10 | 3.38e−09 |
| EPSTI1 | 824.7 | 2.063 | 0.3216 | 6.413 | 1.427e−10 | 3.505e−09 |
| ISG15 | 539.4 | 1.526 | 0.2387 | 6.391 | 1.65e−10 | 3.987e−09 |
| RNF213 | 8932 | 1.041 | 0.1737 | 5.992 | 2.068e−09 | 3.785e−08 |
| IFI6 | 1487 | 1.224 | 0.222 | 5.513 | 3.535e−08 | 4.843e−07 |
| SIGLEC1 | 1696 | 1.8 | 0.3586 | 5.019 | 5.195e−07 | 5.356e−06 |
| IFIT2 | 614.2 | 1.123 | 0.2377 | 4.726 | 2.286e−06 | 1.95e−05 |
| EIF2AK2 | 1963 | 0.559 | 0.121 | 4.62 | 3.833e−06 | 3.082e−05 |
| OAS3 | 2499 | 1.031 | 0.228 | 4.52 | 6.183e−06 | 4.714e−05 |
| SP100 | 2608 | 0.4803 | 0.109 | 4.407 | 1.048e−05 | 7.493e−05 |
| RTP4 | 172.1 | 1.15 | 0.2673 | 4.301 | 1.703e−05 | 0.0001157 |
| LY6E | 5757 | −0.7916 | 0.189 | −4.188 | 2.813e−05 | 0.0001796 |
| SP110 | 1169 | 0.6296 | 0.1538 | 4.094 | 4.239e−05 | 0.0002569 |
| XAF1 | 1654 | 0.9979 | 0.2554 | 3.907 | 9.336e−05 | 0.0005101 |
| MX1 | 1688 | 0.9689 | 0.2548 | 3.803 | 0.0001432 | 0.0007427 |
| STATI | 8279 | 1.073 | 0.2847 | 3.769 | 0.0001641 | 0.0008358 |
| PATL2 | 67.6 | 1.042 | 0.2894 | 3.599 | 0.0003196 | 0.001482 |
| IFIT3 | 1327 | 0.926 | 0.2773 | 3.339 | 0.0008398 | 0.0034 |
| IFI44 | 500.9 | 0.8013 | 0.2739 | 2.926 | 0.003433 | 0.01129 |
| IFI27 | 4515 | 0.8449 | 0.3088 | 2.736 | 0.006216 | 0.01848 |
| LAG3 | 156.4 | 0.9958 | 0.367 | 2.713 | 0.006667 | 0.01964 |
| SPATS2L | 2808 | 0.2462 | 0.09463 | 2.602 | 0.009279 | 0.02596 |
| RSAD2 | 286.7 | 0.7343 | 0.2988 | 2.458 | 0.01398 | 0.03627 |
| HERC5 | 264.5 | 0.4479 | 0.1866 | 2.4 | 0.01639 | 0.04144 |
| CXCL10 | 684.3 | 1.789 | 0.7689 | 2.326 | 0.02001 | 0.04899 |
| LAMP3 | 1275 | 0.6226 | 0.3156 | 1.973 | 0.04853 | 0.1012 |
| IFIT1 | 543 | 0.5069 | 0.2615 | 1.938 | 0.05262 | 0.1082 |
| OAS1 | 1326 | 0.3801 | 0.1992 | 1.908 | 0.05643 | 0.1145 |
| IFI44L | 489.1 | 0.5026 | 0.3174 | 1.583 | 0.1133 | 0.1992 |
| HBB | 1727 | −0.5802 | 0.5911 | −0.9815 | 0.3263 | 0.4558 |
| ISG20 | 678.2 | 0.2456 | 0.32 | 0.7673 | 0.4429 | 0.5708 |
| RGS1 | 946.2 | −0.2499 | 0.5435 | −0.4599 | 0.6456 | 0.7495 |
| PLSCR1 | 1827 | −0.1 | 0.2692 | −0.3716 | 0.7102 | 0.7989 |
| USP18 | 218.4 | 0.02691 | 0.1653 | 0.1628 | 0.8706 | 0.9144 |
| IFI16 | 9461 | 0.02429 | 0.1952 | 0.1244 | 0.901 | 0.9361 |

### Example 2: Gene Expression Analysis in Healthy Volunteers

A study was conducted to assess IFN1 gene expression in healthy volunteers administered a regimen of Compound A. The data set consists of 48 subjects with transcriptome-wide RNA-sequencing data collected for each subject per time-point. For each subject, time points included a pre-dose at day 1, a pre-dose at day 14, and a 4 hour post-day 14 dose. A Wald test was used to assess the association of the various doses at post-dose day 14 versus placebo. To assess potentially off target effects, a secondary transcriptome-wide analysis was performed. Multiple test correction was completed using a Benjamini-Hochberg cutoff of 0.05 within the primary and secondary analyses separately.

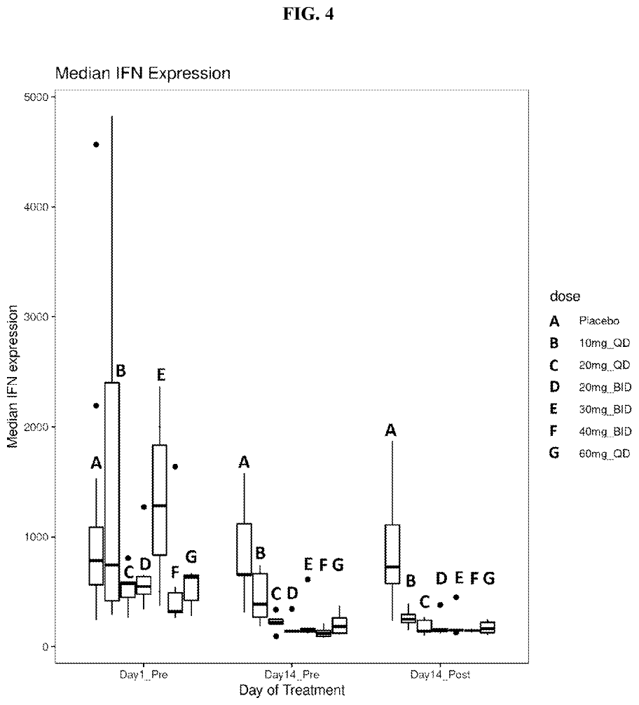

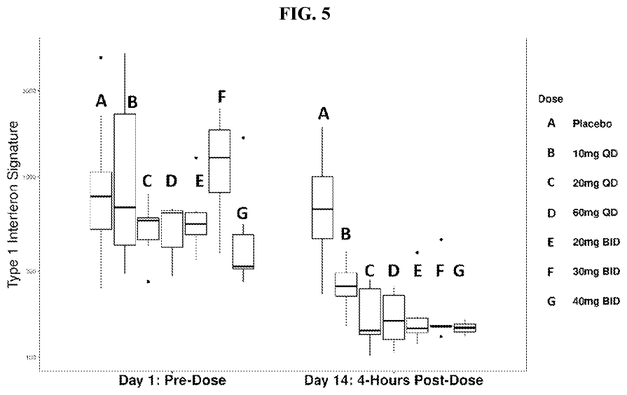

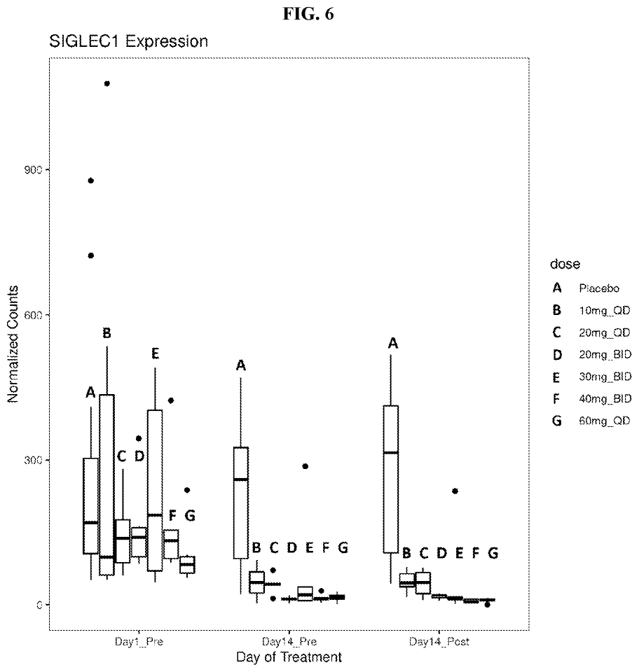

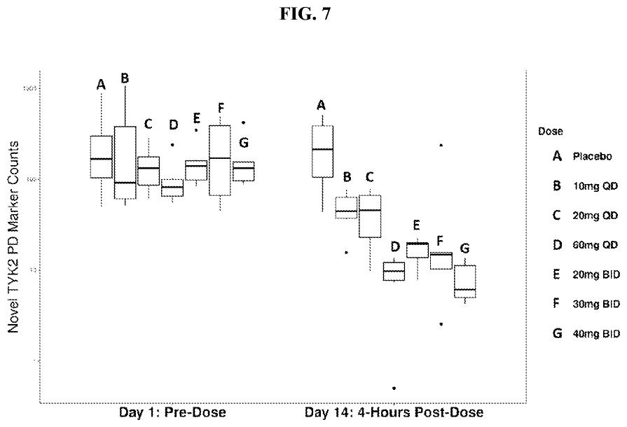

For example FIG. 4 and FIG. 5 show dose-dependent median IFN gene expression levels over time for HERC5, IFI27, IFIT1, RSAD2. As shown in FIG. 4 and FIG. 5, a substantial decrease in IFN expression was observed after 14 days post-treatment with Compound A. FIG. 6 and FIG. 7 shows dose-dependent median IFN gene expression levels over time for SIGLEC1. As shown in FIG. 6 and FIG. 7, SIGLEC1 was decreased after a 14-day regimen of treatment with Compound A.

Table 2, for example, shows IFN gene set results after 14 days of treatment with Compound A versus placebo (P-value based on Wald test). As shown in Table 2, a significant association of Compound A with SIGLEC1 gene expression was observed.

TABLE 2

| Gene | | Mean Base | | BH-Adjusted |
|---|---|---|---|---|
| Symbol | log2FoldChange | Expression | P-value | P-value |
| SIGLEC1 | −3.579 | 124.3 | 3.202e−05 | 0.0004803 |
| MX1 | −1.438 | 2220 | 0.002859 | 0.02144 |
| IFIT1 | −1.676 | 610.3 | 0.01656 | 0.08278 |
| CXCL10 | −1.589 | 12.51 | 0.07494 | 0.281 |
| SPATS2L | −0.5117 | 113.5 | 0.1219 | 0.3318 |
| IFI44L | −1.043 | 1509 | 0.1459 | 0.3318 |
| OAS3 | −0.7134 | 2568 | 0.1549 | 0.3318 |
| ISG15 | −0.8721 | 105.4 | 0.2001 | 0.3496 |
| IFI27 | −1.082 | 33.67 | 0.2098 | 0.3496 |
| IFI44 | −0.3773 | 360.5 | 0.4341 | 0.6512 |
| RSAD2 | −0.4713 | 823.1 | 0.4992 | 0.6807 |
| EPSTI1 | −0.2266 | 579.7 | 0.5786 | 0.7188 |
| ISG20 | 0.08851 | 3376 | 0.623 | 0.7188 |
| OAS1 | −0.1127 | 2267 | 0.7548 | 0.8087 |
| OAS2 | −0.008935 | 6314 | 0.9763 | 0.9763 |

Table 3 shows changes in IFN gene expression in healthy volunteers after 7 days treatment with 40 mg of compound A, twice daily, versus placebo. As shown in Table 3, SIGLEC1 was observed to be the top differentially expressed gene in treated versus placebo.

TABLE 3

| gene_symbol | log2FoldChange | baseMean | pvalue |
|---|---|---|---|
| SIGLEC1 | −3.626 | 148 | 0.0009462 |
| SYN2 | 2.239 | 13.89 | 0.0009678 |
| CETP | 1.503 | 30.81 | 0.00539 |
| SOCS3 | 1.421 | 1032 | 0.007957 |
| HJURP | 1.007 | 40.18 | 0.01959 |
| CHRM3 | −1.521 | 15.87 | 0.02508 |
| OSM | 1.157 | 208.5 | 0.02553 |

Table 4 shows results of a secondary analysis to assess potentially off target effects (contrasting day 14; 4 hour post-treatment versus placebo). As shown in Table 4, no significant off target associations were observed after multiple test corrections.

TABLE 4

| Gene | | Mean Base | | BH-Adjusted |
|---|---|---|---|---|
| Symbol | log2FoldChange | Expression | P-value | P-value |
| SIGLEC1 | −3.579 | 124.3 | 2.651e−05 | 0.5262 |
| IFIT1 | −1.676 | 610.3 | 0.002868 | 1 |
| MX1 | −1.438 | 2220 | 0.004562 | 1 |
| CDNF | 1.185 | 18.41 | 0.004677 | 1 |
| LRP1 | −1.314 | 2334 | 0.005593 | 1 |
| IFI6 | −1.206 | 306.2 | 0.01414 | 1 |
| JPH4 | −1.043 | 23.98 | 0.03419 | 1 |
| LRRC75B | −1.205 | 10.1 | 0.03579 | 1 |
| ENSG00000288643 | −1.541 | 29.29 | 0.03931 | 1 |

### Example 3: SIGLEC1 Whole Blood Gene Expression at Baseline is Predictive of PASI Response in Patients with Psoriasis at 12 Weeks after Treatment with Compound A

Patients with psoriasis were treated with placebo or Compound A, and after 12 weeks of treatment were subject to a Psoriasis Area and Severity Index (PASI) evaluation. Percent change in PASI score was plotted against baseline SIGLEC1 whole blood gene expression.

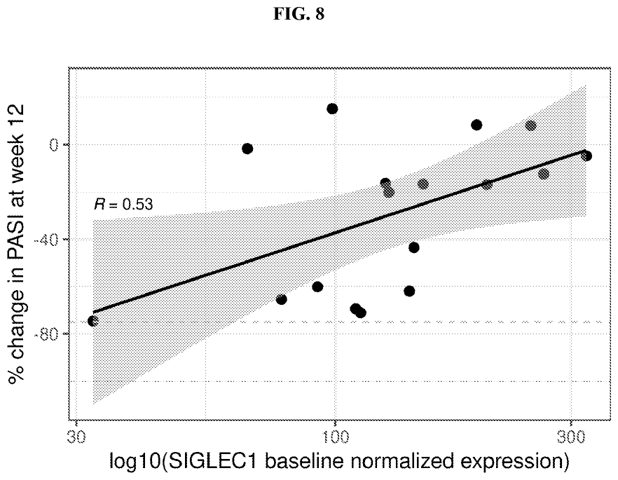

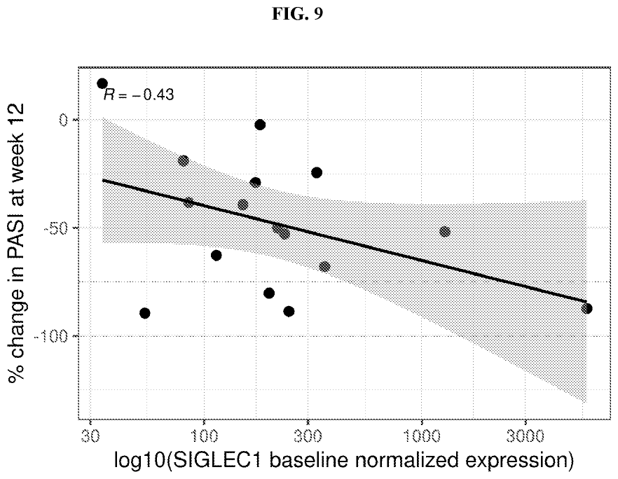

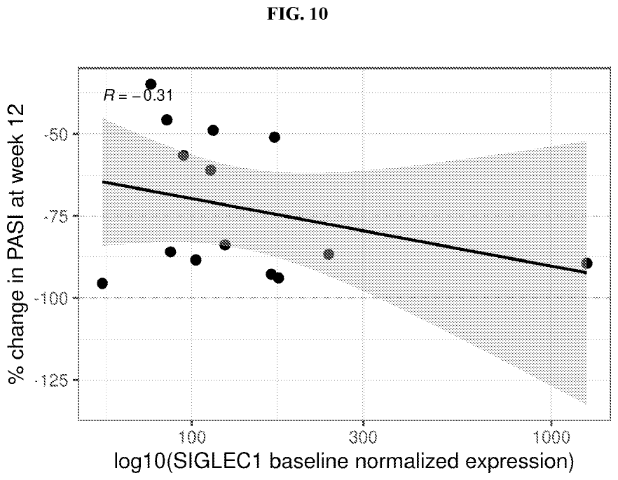

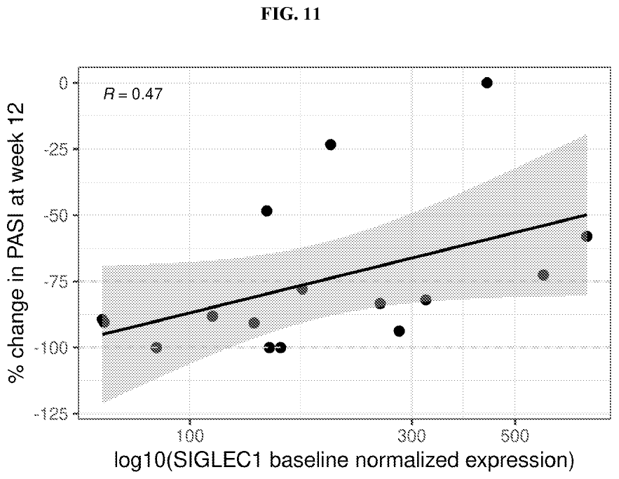

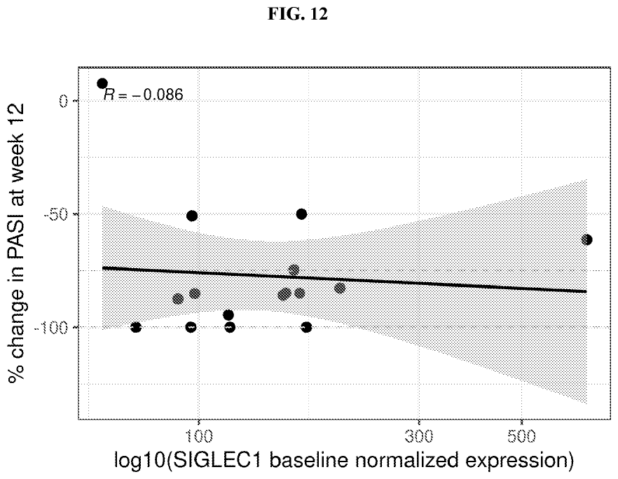

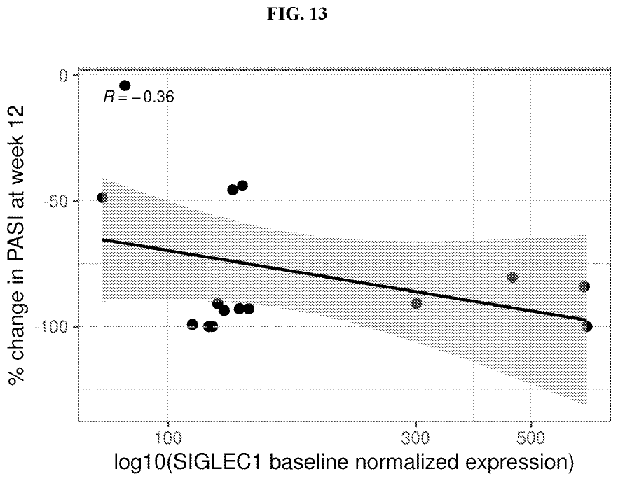

FIG. 8, FIG. 9, FIG. 10, FIG. 11, FIG. 12, and FIG. 13 show percent change in PASI score as a function of baseline SIGLEC1 gene expression (log 10 scale) in patients with psoriasis after 12 weeks of treatment with placebo, Compound A (10 mg, once daily), Compound A (20 mg, once daily), Compound A (40 mg, once daily), Compound A (20 mg, twice daily), and Compound A (40 mg, twice daily), respectively. As shown in FIG. 8 to FIG. 13, elevated baseline SIGLEC1 gene expression can be correlated with a greater change in PASI response.

### Example 4: SIGLEC1 Expression in Patients with Skin Disorders Vs. Healthy Subjects

Publicly available gene expression data sets were analyzed to test for statistically significant upregulation of SIGLEC1 in patients with skin disorders versus healthy subjects. The results are shown in Table 5. As shown in Table 5, the changes in SIGLEC1 were significant, with p-value adjusted for multiple testing <0.1 and enriched in patients with skin disorders compared to healthy subjects.

TABLE 5

| Disease/Disorder | Sample | log2FoldChange | Padj |
|---|---|---|---|
| systemic lupus erythematosus | blood | 3.150892461 | 4.49E−22 |
| cutaneous lupus erythematous | skin | 2.157144433 | 3.99E−06 |
| hidradenitis suppurativa skin | skin | 1.783698872 | 5.96E−06 |
| subacute cutaneous lupus | skin | 2.197338045 | 0.001234863 |
| erythematosus | | | |
| dermatomyositis | muscle | 1.855137889 | 0.002381401 |
| juvenile dermatomyositis | blood | 2.903796856 | 0.038502825 |
| chronic cutaneous lupus | skin | 2.028622672 | 0.038829559 |
| erythematous | | | |
| acute cutaneous lupus | skin | 2.198402607 | 0.0545622 |
| erythematous | | | |
| rheumatoid arthritis | blood | 0.317236946 | 0.091346071 |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating a patient suffering from an auto-immune skin disease and having elevated Type I IFN induced sialic acid-binding immunoglobulin-like lectin1 (SIGLEC1), and in need of treatment, comprising administering to the patient an effective amount of a TYK2 inhibitor.

2. The method of claim 1, wherein a lesional skin sample from the patient before administration of the TYK2 inhibitor has elevated Type I IFN-induced sialic acid-binding immunoglobulin-like lectin1 (SIGLEC1) expression.

3. The method of claim 2, wherein the elevated Type I IFN induced sialic acid-binding immunoglobulin-like lectin1 (SIGLEC1) expression is elevated as compared to the SIGLEC1 expression in the whole blood of the patient, or as compared to a patient not suffering from the auto-immune skin disease.

4. The method of claim 1, wherein a biological sample from the patient before administration of the TYK2 inhibitor has increased IFN-1 gene expression.

5. The method of claim 4, wherein the increased IFN-1 gene expression is one or more of increased gene expression of: OAS3, OAS2, CD₈A, EPSTI1, BST2, RNF213, EIF2AK2, IFIT2, IFIT3, SP100, SP110, LY6E, MX1, IFI6, RTP4, XAF1, PATL2, STAT1, and SIGLEC1.

6. A method of treating a dermatological disorder in a patient having elevated Type I IFN-induced sialic acid-binding immunoglobulin-like lectin1 (SIGLEC1) expression, and in need of treatment, comprising administering to the patient an effective amount of a TYK2 inhibitor, and thereby upon administration reducing the expression of SIGLEC1 in the patient.

7. A method of reducing expression of SIGLEC1 protein or gene in a cell, comprising contacting the cell with an agent that reduces TYK2 gene expression or reduces TYK2 in the cell, thereby reducing the expression of SIGLEC1.

8. The method of claim 7, wherein the cell is a skin cell.

9. A method of treating a subject suffering from an auto-immune skin disease, and in need of treatment, comprising:
providing a biological sample from the subject;
assaying gene expression of one or more IFN-1 genes in the biological sample;
determining the expression value of the IFN-1 gene(s); and
if the expression value is above a threshold value, administering a TYK2 inhibitor to the patient.

10. The method of claim 9, wherein the IFN-1 gene is one or more of: OAS3, OAS2, CD₈A, EPSTI1, BST2, RNF213, EIF2AK2, IFIT2, IFI6, and SIGLEC1.

11. The method of claim 9 or 10, wherein the biological sample is the subject's blood or tissue.

12. The method of any one of claims 1-11, wherein the TYK2 inhibitor is selected from the group consisting of GLPG3121, GLPG3667, VTX-958, ICP-332, BGB-23339, 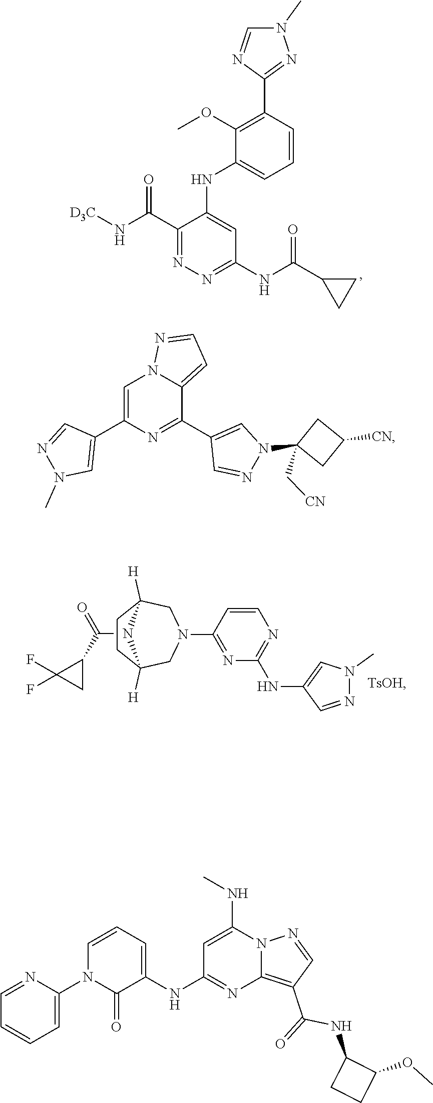 [SMILES: [2H]C([2H])([2H])NC(=O)c1nnc(NC(=O)C2CC2)cc1Nc1cccc(-c2ncn(C)n2)c1OC ; Cn1cc(-c2cn3nccc3c(-c3cnn([C@]4(CC#N)C[C@@H](C#N)C4)c3)n2)cn1 ; Cn1cc(Nc2nccc(N3C[C@H]4CC[C@@H](C3)N4C(=O)[C@@H]3CC3(F)F)n2)cn1 ; CNc1cc(Nc2cccn(-c3ccccn3)c2=O)nc2c(C(=O)N[C@@H]3CC[C@H]3OC)cnn12]
and

13. The method of any one of claims 1-12, wherein the TYK2 inhibitor is represented by: 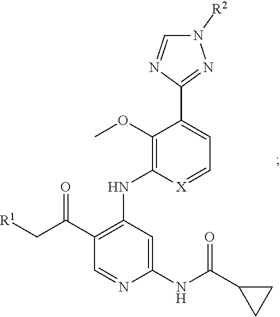
or a pharmaceutically acceptable salt thereof;
wherein:
X is CH or N; and
R¹ and R² are independently for each occurrence selected from CH₃ and CD₃.

14. The method of any one of claims 12-13, comprising orally administering or topically administering the compound to the patient.

15. The method of any one of claims 1-14, wherein the auto-immune skin disease or dermatological disorder is one or more of: systemic sclerosis, Sjogren's syndrome, cutaneous lupus erythematosus, systemic lupus erythematosus with skin manifestations, psoriasis, dermatomyositis, hidradenitis superativa, vitiligo, and Behcet's Disease.

16. The method of any one of claims 1-14, wherein the auto-immune skin disease or dermatological disorder is one or more of: vasculitis, Pemphigus disease, alopecia, atopic dermatitis, epidermolysis bullosa, bullous pemphigoid, and dermatitis herpetiformis.
